(19) United States
(12) Patent Application Publication
(10) Pub. No.: US 2024/0379235 A1
(43) Pub. Date: Nov. 14, 2024

(54) MULTI-OMIC PREDICTION MODEL FOR METASTATIC PROSTATE CANCER

(71) Applicant: University of Utah Research Foundation, Salt Lake City, UT (US)

(72) Inventors: Manish Kohli, Salt Lake City, UT (US); Robert Michael Kirby, Salt Lake City, UT (US); Shikai Fang, Salt Lake City, UT (US); Shandian Zhe, Salt Lake City, UT (US)

(21) Appl. No.: 18/661,495

(22) Filed: May 10, 2024

Related U.S. Application Data
(60) Provisional application No. 63/466,029, filed on May 12, 2023.

(51) Int. Cl. G16H 50/20

(52) U.S. Cl. CPC G16H 50/20

(57) ABSTRACT

Disclosed is a method for predicting a clinical outcome in a metastatic prostate cancer patient based on measurements of multi-omic features of the patient. A weighting model is selected based on the state of the metastatic prostate cancer (a metastatic hormone sensitive prostate cancer (mHSPC) state or a metastatic castrate resistant prostate cancer (mCRPC) state) and on a clinical outcome of interest. The weighting model maps weights to a series of features (including genetic markers and lipid markers) with respect to the clinical outcome of interest. The weights are generated using a machine learning process that utilizes a training dataset that associates the features of multiple historic metastatic prostate cancer patients with recorded clinical outcomes of those patients. A predictive model generates a probability score for the patient indicating the likelihood of occurrence of the clinical outcome of interest.

### CROSS-REFERENCE TO RELATED APPLICATIONS

This application claims priority to and the benefit of U.S. Provisional Application No. 63/466,029, filed May 12, 2023 and titled “Machine Learning Models in Metastatic Prostate Cancer,” the entirety of which is incorporated herein by reference.

### BACKGROUND

Prostate cancer is a leading cause of cancer death in males in the western world. Metastatic prostate cancer is sub-divided into an earlier metastatic hormone-sensitive prostate cancer (mHSPC) state and a more progressed metastatic castrate resistant prostate cancer (mCRPC) state. Androgen deprivation therapy (ADT) is the primary means of treating mHSPC. When this fails, the cancer typically progresses to the mCRPC state.

Both mHSPC and mCRPC are heterogeneous in clinical behavior, and patients in these states can either progress quickly or slowly. Response to treatments given in these states can similarly be short or long-lasting. An increasing number of treatment options for managing mHSPC and mCRPC states are now available. However, conventionally, there are no specific molecular multi-omic alterations that are predictive for response to ADT or prognostic for survival.

Accordingly, there is an ongoing need for improved methods for predicting clinical outcomes in metastatic prostate cancer patients. Improved predictions of clinical outcomes can lead to more effective and/or more personalized patient treatment plans.

### SUMMARY

Disclosed is a method for predicting a clinical outcome in a metastatic prostate cancer patient based on measurements of multi-omic features of the patient. The multi-omic features include genetic markers and lipid markers.

In one embodiment, a weighting model is selected based on the state of the metastatic prostate cancer (an mHSPC state or an mCRPC state) and on a clinical outcome of interest (e.g., survival in the mHSPC state, ADT failure in the mHSPC state, early death in the mCRPC state, or late death in the mCRPC state). The weighting model maps weights to a series of features (including genetic markers and lipid markers) with respect to the clinical outcome of interest. The weights are generated using a machine learning process that utilizes a training dataset that associates the features of multiple historic metastatic prostate cancer patients with recorded clinical outcomes of those patients.

A predictive model generates a probability score (PS) for the patient indicating the likelihood of occurrence of the clinical outcome of interest. The predictive model is defined by:

$$PS = \frac{1}{1 + exp ( - {\sum }_{n = 1}^{N} {w}_{n} {x}_{n} )}$$

wherein PS represents the likelihood of occurrence of the clinical outcome of interest, N is the number of different features used in the determination of PS, wₙ are weights for each nth feature as provided by the selected weighting model, and xₙ are the measured values of the patient for each nth feature.

The PS can beneficially be used to inform medical practitioners about the likelihood of certain clinical outcomes. Medical practitioners can use this information to develop or modify patient treatment plans with the aim of improving patient outcomes, better personalizing treatment to the individual patient, and/or increasing the overall effectiveness of patient care.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the disclosure will become apparent and more readily appreciated from the following description, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates an example workflow for identifying genetic marker and lipid marker features and using a machine learning process to generate weights for the features for use in a predictive model to provide probability scores for a clinical outcome of interest;

FIG. 2 illustrates a bin-split binarization method for binarizing lipid marker plasma levels;

FIG. 3 illustrates an example approach for calculating a probability score for the clinical outcome of interest using a subset of features used in the full training dataset; and

FIGS. 4A-4D illustrate test accuracy as a function of the ratio of selected features (N) to total candidate features of the training dataset, showing that high accuracy is maintained even when as low as 50% of the features of the original training dataset are utilized.

### DETAILED DESCRIPTION

### Predictive Model Overview

In an example method for predicting a clinical outcome in a metastatic prostate cancer patient, a weighting model is selected based on the state of the metastatic prostate cancer (an mHSPC state or an mCRPC state) and on a clinical outcome of interest (e.g., survival in the mHSPC state, ADT failure in the mHSPC state, early death in the mCRPC state, or late death in the mCRPC state). The weighting model maps weights to a series of features (including genetic markers and lipid markers) with respect to the clinical outcome of interest. The weights are generated using a machine learning process that utilizes a training dataset that associates the features of multiple historic metastatic prostate cancer patients with recorded clinical outcomes of those patients.

The weighting model can be used in conjunction with a predictive model configured to generate a probability score (PS) for the patient. The PS indicates the likelihood that the clinical outcome of interest will occur. The predictive model is defined by:

$$PS = \frac{1}{1 + exp ( - {\sum }_{n = 1}^{N} {w}_{n} {x}_{n} )}$$

wherein PS represents the likelihood of occurrence of the clinical outcome of interest, N is the number of different features used in the determination of PS, wₙ are weights for each nth feature as provided by the selected weighting model, and xₙ are the measured values of the patient for each nth feature. Optionally, the PS can be compared to a pre-set threshold probability, such as a probability of 0.5 (i.e., the probability of an outcome with a coin flip) to arrive at a binary prediction probability for the clinical outcome of interest.

As discussed in more detail below, N need not be equal to the total number of features included in the training dataset of the weighting model. That is, N can represent a subset of features used in the training dataset. Beneficially, the model can utilize N as a subset of total features without substantially affecting the accuracy of the predictive model relative to when the full set of features is used. For example, so long as the training dataset has sufficient number of features (e.g., at least about 50 features), N can be lowerable to as low as 50% of the total number of features in the training dataset without substantial degradation in accuracy of the resulting PS (e.g., without suffering more than 10% loss in accuracy relative to using the full set of features from the training dataset). The N number of features used in the determination of PS can be the N number of features with the highest effect sizes as determined by the machine learning process.

Failure in the mHSPC state can be defined as failure within a predetermined time period (e.g., 6 months) following initiation of ADT. Early death in the mCRPC state can be defined as being before a predefined threshold or as being within a predetermined bottom quantile or set of quantiles for survival time following progression to the mCRPC state. Such quantiles can be determined from the training dataset. Similarly, late death in the mCRPC state can be defined as being beyond a predefined threshold or as being within a predetermined top quantile or set of quantiles for survival time following progression to the mCRPC state. Such quantiles can be determined from the training dataset. For example, the lower tertile of survival times for mCRPC patients could be used to define the cutoff for early death, and the upper tertile could be used to define the threshold for late death.

The patient's measured values for the features can be obtained in any suitable fashion. In presently preferred embodiments, the features include genetic and lipid markers that can be readily determined from a straightforward blood (e.g., plasma) sample from the patient. That is, the lipid markers can comprise plasma lipid levels and the genetic markers can comprise circulating tumor DNA (ctDNA) markers, which can be determined from patient blood using methods known to those of skill in the art.

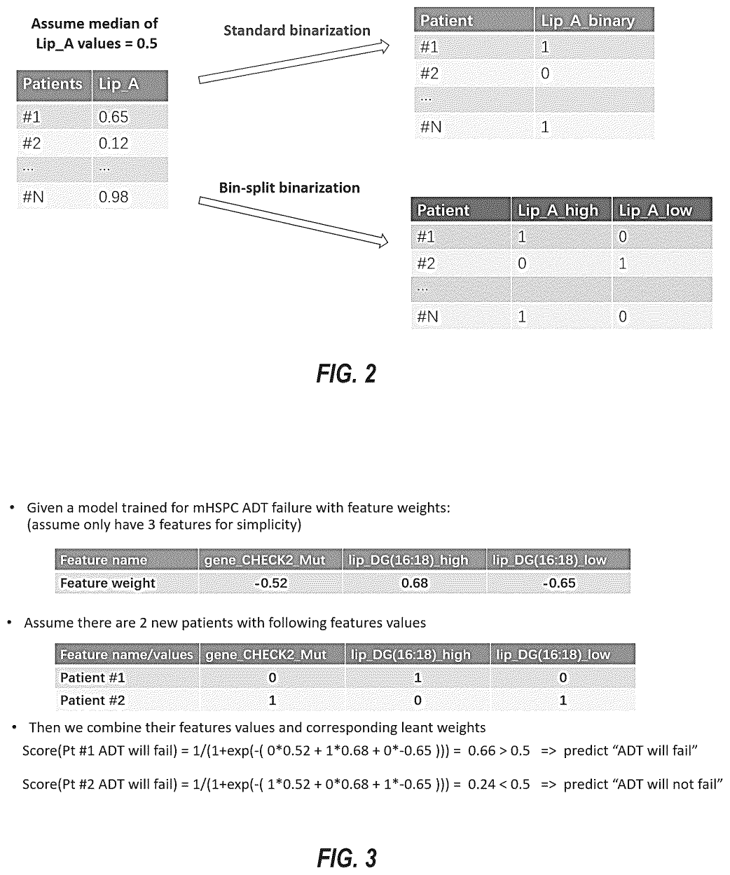

For purposes of the predictive model, the genetic markers can be binarized as present or absent. Because levels of the lipid markers can exist on a spectrum, these too can be binarized according to high-level or low-level. However, as described in more detail below, certain benefits can be obtained by using a bin-split binarization process in which each lipid marker measurement is mapped to both a high-level binary feature and a low-level binary feature, rather than simply binarized as high/low. See FIG. 2 and corresponding description. For each lipid marker, a predetermined threshold level (e.g., the median level of that lipid marker in the training dataset) can be used as a cutoff between the high-level binary feature and the low-level binary feature for that lipid marker.

Any suitable set of genetic markers can be utilized in the set of features of the weighting and predictive models. In certain embodiments disclosed herein, the genetic markers comprise one or more of ATM, BRCA1, BRCA2, or CHEK2 when the patient is in the mHSPC state and/or comprise one or more of TP53, RB1 loss, or AR amplification when the patient is in the mCRPC state. Other genetic markers may additionally or alternatively be incorporated as features of the weighting and predictive models. Other genetic markers may be later discovered as relevant to metastatic prostate cancer, for example, and such can be readily incorporated into the models.

Similarly, any suitable set of lipid markers can be utilized in the set of features of the weighting and predictive models. In certain embodiments disclosed herein, the lipid markers comprise one or more of ceramide (Cer) species, diacylglycerol (DG) species, and triacylglycerol (TG) species when the patient is in the mHSPC state and/or comprise one or more of Cer species, sphingosine (Sph) species, and acylcarnitine (Acy) species when the patient is in the mCRPC state. More detailed examples of lipid markers are provided in the tables disclosed below. Other lipid markers may additionally or alternatively be incorporated as features of the weighting and predictive models. Other lipid markers may be later discovered as relevant to metastatic prostate cancer, for example, and such can be readily incorporated into the models.

The weighting model can utilize any suitable regression, classification, and/or machine learning method to determine and map the weights to their respective features. For example, when a machine learning process is utilized, the machine learning process can include one or more of: logistic regression, optionally with elastic-net regularization; kernel support vector machines (kernel-SVM); or Gaussian process regression (GPR). In a presently preferred embodiment, the machine learning process utilizes logistic regression with elastic-net regularization. Performance of the machine learning process can be evaluated by determining, for example, the area under the curve (AUC) of a receiver operating characteristic (ROC) curve. This can be calculated as a concordance index (C-index). The results of the machine learning process can also be analyzed for accuracy, precision, recall, and/or sensitivity.

The PS can beneficially be used to inform medical practitioners about the likelihood of certain clinical outcomes. Medical practitioners can use this information to develop or modify patient treatment plans with the aim of improving patient outcomes, better personalizing treatment to the individual patient, and/or increasing the overall effectiveness of patient care.

For example, considering the model for ADT failure in the mHSPC state, if the PS shows a high probability (e.g., higher than 50% chance) that ADT will fail within a short period of time (e.g., 6 months) after starting treatment, medical practitioners and patients can discuss adding chemotherapy and/or other treatments in addition or as an alternative to ADT, to potentially achieve remission for a longer time. On the other hand, if the PS indicates a low probability (e.g., lower than 50% chance) that ADT will fail within the initial treatment time period (e.g., 6 months), medical practitioners and patients may be in a more informed position to avoid or minimize chemotherapy and/or other treatments, which can have harmful side effects.

In another example, considering the model for early death or the model for late death/long survival in the mCRPC state, if the PS indicates a high probability that the patient will be a long-term survivor, the medical practitioners and patient can be better informed of the tradeoffs surrounding additional treatments, which can be physically and financially demanding. Such information may, for example, minimize unnecessary use of aggressive treatments and help the patient avoid toxicities and extra burdens that are unlikely to promote longevity and quality of life. On the other hand, if the PS indicates a high probability of early death and/or a low probability of being a long-term survivor, the patient and medical practitioners can better understand that aggressive treatments may be worthwhile despite the physical and financial burdens.

### Working Examples

### Patient Cohort

A real world prospectively collected and retrospectively analyzed hospital-based cohort of metastatic prostate cancer patients, in mHSPC and mCRPC states, were utilized. Previously, this cohort's plasma cfDNA alterations using next gen-sequencing (NGS) and lipid species using plasma-based liquid chromatography mass spectrometry (LC-MS) were determined. The number of lipid species and genomic alteration features for building multi-omic classifiers was 722.

The metastatic prostate cancer cohort was enrolled between September 2009 and January 2013 after obtaining a written consent and Institutional Review Board approval (Mayo IRB #09-001889). Clinical follow-up for this cohort in the current study was extended to Mar. 1, 2022. Progression on ADT, labelled as ADT failure for mHSPC patients, was defined as time from initiating hormone therapy for metastatic state to development of castrate resistance, and survival was calculated as time from diagnosis of mHSPC state to death at the date of cut off for analysis (Mar. 1, 2022). Patients in mHSPC state who did not have a death event at the time of data analysis were censored. In the mHSPC cohort, we labelled patients who failed ADT within 6 months after initiation as early failure.

The survival time of patients with mCRPC was determined from the date of castrate resistance to the date of death at the time of analysis (Mar. 1, 2022). The range of survival time of the mCRPC patients was divided into tertiles. Thus, patients in the bottom 33% of survival time (who died within 618 days after initial progression to mCRPC) were classified as belonging to the poor prognosis group, while those who were still alive in the top 33% of the survival range at or after 1500 days of initial progression to mCRPC were grouped as exceptional survivors.

Four multi-omic classifier-building tasks were pursued using machine learning methods, two in mHSPC state and two in mCRPC state. For mHSPC patients, we utilized a machine-learning classifier model that predicted mHSPC survival and a machine learning-based classifier model that predicted early failure of ADT. In the mCRPC state, we developed a multi-omic classifier model that predicted patients with poor prognosis (death within 618 days of turning castration-resistant) and a model that predicted exceptional survival as defined by long-term alive state at 1500 days or more after turning castration-resistant.

### Feature Selection Process

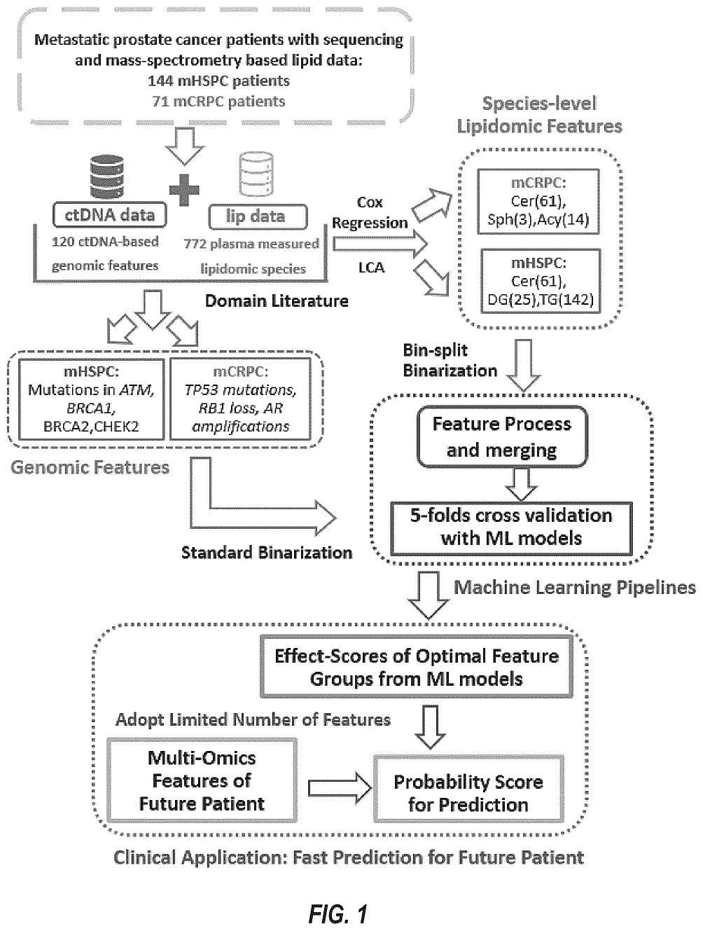

The overall methodology workflow is illustrated in FIG. 1. Informative features were identified from previously published results. To start, we considered 120 ctDNA-based gene alterations and 722 plasma-measured lipidomic species. Based on previous results that confirm specific cfDNA-based signatures in these 120 genes to have predictive and prognostic value in metastatic prostate cancer, we adopted these alterations to proceed to the next step. We then selected mutations in ATM, BRCA1, BRCA2, and CHEK2 for patients in the mHSPC state, and mutations in TP53, RB1 loss, and AR amplification for patients in the mCRPC state, for integration with lipidomic features as potential classifiers.

For the 772-lipidomic features, we applied Cox regression and latent component analysis (LCA) to identify candidate lipid species. This resulted in the selection of 61 ceramide (Cer) species, 25 diacylglycerol (DG) species, and 142 triacylglycerol (TG) species for patients in the mHSPC state, and 61 ceramide (Cer) species, 3 sphingosine (Sph) species, and 14 acylcarnitine (Acy) species for patients in the mCRPC state. A selection of the lipid marker features included in the modeling is provided in Table 1:

TABLE 1
Lipid Marker Features Included in Modeling

| | | | |
|---|---|---|---|
| Sph(d16:1) | Cer(m18:1/24:0) | TG(54:2) [SIM] | TG(54:1) [NL-18:1] |
| Sph(d18:1) | Cer(m18:1/24:1) | TG(54:3) [SIM] | TG(54:2) [NL-18:0] |
| Sph(d18:2) | AC(12:0) | TG(54:4) [SIM] | TG(54:2) [NL-20:1] |
| Cer(d16:1/16:0) | AC(13:0) | TG(54:5) [SIM] | TG(54:3) [NL-18:1] |
| Cer(d16:1/18:0) | AC(14:0) | TG(54:6) [SIM] | TG(54:3) [NL-18:2] |
| Cer(d16:1/20:0) | AC(14:1) | TG(54:7) [SIM] | TG(54:4) [NL-18:2] |
| Cer(d16:1/22:0) | AC(14:2) | TG(56:6) [SIM] | TG(54:4) [NL-20:3] |
| Cer(d16:1/23:0) | AC(15:0) (a) | TG(56:7) [SIM] | TG(54:5) [NL-18:3] |
| Cer(d16:1/24:0) | AC(15:0) (b) | TG(56:8) [SIM] | TG(54:5) [NL-20:4] |
| Cer(d16:1/24:1) | AC(16:0) | TG(56:9) [SIM] | TG(54:6) [NL-18:3] |
| Cer(d17:1/16:0) | AC(16:1) | TG(58:10) [SIM] | TG(54:6) [NL-20:4] |
| Cer(d17:1/18:0) | AC(17:0) (a) | TG(58:8) [SIM] | TG(54:6) [NL-20:5] |
| Cer(d17:1/20:0) | AC(17:0) (b) | TG(58:9) [SIM] | TG(54:6) [NL-22:6] |
| Cer(d17:1/22:0) | AC(18:0) | TG(48:0) [NL-16:0] | TG(54:7) [NL-20:5] |
| Cer(d17:1/23:0) | AC(18:1) | TG(48:0) [NL-18:0] | TG(54:7) [NL-22:6] |
| Cer(d17:1/24:0) | AC(18:2) | TG(48:1) [NL-16:1] | TG(56:6) [NL-20:4] |
| Cer(d17:1/24:1) | DG(14:0_16:0) | TG(48:1) [NL-18:1] | TG(56:6) [NL-22:5](a) |
| Cer(d18:1/14:0) | DG(16:0_16:0) | TG(48:2) [NL-14:0] | TG(56:6) [NL-22:5](b) |
| Cer(d18:1/16:0) | DG(16:0_16:1) | TG(48:2) [NL-14:1] | TG(56:7) [NL-20:4] |
| Cer(d18:1/17:0) | DG(14:0_18:2) | TG(48:2) [NL-16:1] | TG(56:7) [NL-20:5] |
| Cer(d18:1/18:0) | DG(16:0_18:1) | TG(48:2) [NL-18:2] | TG(56:7) [NL-22:5](a) |
| Cer(d18:1/19:0) | DG(16:1_18:1) | TG(48:3) [NL-14:0] | TG(56:7) [NL-22:5](b) |
| Cer(d18:1/20:0) | DG(16:0_18:2) | TG(48:3) [NL-16:1] | TG(56:7) [NL-22:6] |
| Cer(d18:1/21:0) | DG(18:0_18:1) | TG(48:3) [NL-18:3] | TG(56:8) [NL-20:4] |
| Cer(d18:1/22:0) | DG(18:1_18:1) | TG(49:1) [NL-16:1] | TG(56:8) [NL-20:5] |
| Cer(d18:1/23:0) | DG(18:0_18:2) | TG(49:1) [NL-17:1] | TG(56:8) [NL-22:6] |
| Cer(d18:1/24:0) | DG(18:1_18:2) | TG(50:0) [NL-18:0] | TG(56:9) [NL-22:6] |
| Cer(d18:1/24:1) | DG(18:2_18:2) | TG(50:1) [NL-14:0] | TG(58:10) [NL-22:6] |
| Cer(d18:1/26:0) | DG(18:1_18:3) | TG(50:1) [NL-16:0] | TG(58:8) [NL-22:6] |
| Cer(d18:2/14:0) | DG(16:0_20:4) | TG(50:1) [NL-18:1] | TG(58:9) [NL-22:6] |
| Cer(d18:2/16:0) | DG(18:1_20:3) | TG(50:2) [NL-14:0] | TG(O-50:1) [SIM] |
| Cer(d18:2/17:0) | DG(18:0_20:4) | TG(50:2) [NL-16:1] | TG(O-50:2) [SIM] |
| Cer(d18:2/18:0) | DG(18:1_20:4) | TG(50:2) [NL-18:1] | TG(O-50:3) [SIM] |
| Cer(d18:2/20:0) | DG(16:0_22:5) | TG(50:2) [NL-18:2] | TG(O-52:0) [SIM] |
| Cer(d18:2/21:0) | DG(18:2_20:4) | TG(50:3) [NL-14:0] | TG(O-52:1) [SIM] |
| Cer(d18:2/22:0) | DG(16:0_22:6) | TG(50:3) [NL-14:1] | TG(O-52:2) [SIM] |
| Cer(d18:2/23:0) | DG(18:1_20:5) | TG(50:3) [NL-16:1] | TG(O-54:2) [SIM] |
| Cer(d18:2/24:0) | DG(18:1_22:5) | TG(50:3) [NL-18:2] | TG(O-54:3) [SIM] |
| Cer(d18:2/24:1) | DG(18:0_22:6) | TG(50:3) [NL-18:3] | TG(O-54:4) [SIM] |
| Cer(d18:2/26:0) | DG(18:1_22:6) | TG(50:4) [NL-14:0] | TG(O-50:1) [NL-15:0] |
| Cer(d19:1/16:0) | DG(18:2_22:6) | TG(50:4) [NL-18:3] | TG(O-50:1) [NL-16:0] |
| Cer(d19:1/18:0) | TG(48:0) [SIM] | TG(50:4) [NL-20:4] | TG(O-50:1) [NL-17:1] |
| Cer(d19:1/20:0) | TG(48:1) [SIM] | TG(51:0) [NL-16:0] | TG(O-50:1) [NL-18:1] |
| Cer(d19:1/22:0) | TG(48:2) [SIM] | TG(51:1) [NL-17:0] | TG(O-50:2) [NL-16:1] |
| Cer(d19:1/23:0) | TG(48:3) [SIM] | TG(51:2) [NL-15:0] | TG(O-50:2) [NL-18:1] |
| Cer(d19:1/24:0) | TG(49:1) [SIM] | TG(51:2) [NL-17:0] | TG(O-50:2) [NL-18:2] |
| Cer(d19:1/24:1) | TG(50:0) [SIM] | TG(51:2) [NL-17:1] | TG(O-50:3) [NL-18:2] |
| Cer(d19:1/26:0) | TG(50:1) [SIM] | TG(52:1) [NL-18:0] | TG(O-52:0) [NL-16:0] |
| Cer(d20:1/22:0) | TG(50:2) [SIM] | TG(52:1) [NL-18:1] | TG(O-52:1) [NL-16:0] |
| Cer(d20:1/23:0) | TG(50:3) [SIM] | TG(52:2) [NL-16:0] | TG(O-52:1) [NL-18:1] |
| Cer(d20:1/24:0) | TG(50:4) [SIM] | TG(52:2) [NL-18:2] | TG(O-52:2) [NL-16:0] |
| Cer(d20:1/24:1) | TG(51:0) [SIM] | TG(52:3) [NL-16:1] | TG(O-52:2) [NL-17:1] |
| Cer(d20:1/26:0) | TG(51:1) [SIM] | TG(52:3) [NL-18:2] | TG(O-52:2) [NL-18:1] |
| Cer(m18:0/20:0) | TG(51:2) [SIM] | TG(52:4) [NL-16:1] | TG(O-54:2) [NL-17:1] |
| Cer(m18:0/22:0) | TG(52:1) [SIM] | TG(52:4) [NL-18:2] | TG(O-54:2) [NL-18:1] |
| Cer(m18:0/23:0) | TG(52:2) [SIM] | TG(52:4) [NL-18:3] | TG(O-54:3) [NL-17:1] |
| Cer(m18:0/24:0) | TG(52:3) [SIM] | TG(52:5) [NL-18:3] | TG(O-54:3) [NL-18:1] |
| Cer(m18:0/24:1) | TG(52:4) [SIM] | TG(52:5) [NL-20:4] | TG(O-54:4) [NL-17:1] |
| Cer(m18:1/18:0) | TG(52:5) [SIM] | TG(52:5) [NL-20:5] | TG(O-54:4) [NL-18:2] |
| Cer(m18:1/20:0) | TG(53:2) [SIM] | TG(53:2) [NL-17:1] | — |
| Cer(m18:1/22:0) | TG(54:0) [SIM] | TG(53:2) [NL-18:1] | — |
| Cer(m18:1/23:0) | TG(54:1) [SIM] | TG(54:0) [NL-18:0] | — |

Abbreviations:
Sph = Sphingoid,
Cer = Ceramide,
AC = Acylcarnitine,
DG = Diacylglycerol,
TG = triacylglycerol

For feature processing, genomic alterations were transformed into binary variables (present/absent). Lipidomic species' measurements, being continuous features, were binarized using a novel approach called bin-split binarization, which differs from conventional mean-split binarization methods which categorize continuous features based on the mean/median levels. The bin-split binarization maps each numerical lipid feature to two separate binary features-high-level-lipid and low-level-lipid, allowing for a finer-grained analysis of the effects of different lipid levels.

An illustration of the bin-split binarization is shown in FIG. 2. The bin-split binarization approach promotes understanding of the different effects of high-level and low-level cases within each candidate lipid molecule. Since two binary features are used, we can learn the two different sets of weights for each lipid marker (one for the high-level and one for the low-level) through our machine learning models. Standard binarization provides only one learned binarized feature weight, which would force the high-level and low-level of each lipid marker to have a strict inversely proportional relationship. Of course, a strict inversely proportional relationship may not be accurate, so decoupling the weighting of the high-level and low-level features for each lipid marker allows a more granular weighting scheme.

### Machine Learning Methods

We evaluated several machine learning techniques, including logistic regression with and without elastic-net regularization, kernel support vector machines (kernel-SVM), and Gaussian process regression (GPR) with multiple kernel configurations. After identifying the optimal model and to mitigate overfitting, we trained and evaluated the model using a standard five-fold cross-validation approach. All mHSPC and mCRPC patients were divided into five equal groups, with one group used to test the model while the other four were used to train the model. This process was repeated five times to ensure each group was included in the model evaluation. After all five iterations, the average of the recorded accuracy scores is taken as the final test score, providing a comprehensive evaluation of the performance of the models. All models were trained using gradient-based optimization algorithms.

The performance of each classifier was evaluated using five metrics: area under the curve (AUC), calculated as Concordance index or C-index; accuracy; precision; recall; and sensitivity. Monte-Carlo five-fold cross-validation was used to determine the mean and 0.95 confidence interval for each metric.

AUC is a measure of the overall performance of a binary classification model. It represents the area under the receiver operating characteristic (ROC) curve and ranges from 0.5 (random classification) to 1.0 (perfect classification). A higher AUC indicates better model performance. AUC is equal to the Concordance index or C-index under the binary classification case. Accuracy is the proportion of correctly classified samples out of the total number of samples. It is calculated as the sum of true positives and true negatives divided by the total number of samples. Precision is the proportion of true positives among all predicted positives. It is calculated as the number of true positives divided by the sum of true positives and false positives. Recall (also known as sensitivity) is the proportion of true positives among all actual positives. It is calculated as the number of true positives divided by the sum of true positives and false negatives. Specificity is the proportion of true negatives among all actual negatives. It is calculated as the number of true negatives divided by the sum of true negatives and false positives.

### Determination of Feature Effects

We analyzed the top 20 features which were observed to have high positive or negative weights for each clinical outcome and then calculated their relative ratios based on the raw weight values over the feature groups yielding the most optimal performance. The relative effect ratio indicates the relative importance of each predictive or protective feature, based on the raw feature weights produced by the model.

The formula used for computing the relative effect ratio involves dividing each positive or negative feature weight by the sum of all positive or negative feature weights. This produces a ratio that measures the relative significance of each feature in the model. Computing the relative effect ratios can identify which features are the most important outcome predictors, and which features may be less significant or even redundant. This can be used to improve the model by removing or adjusting redundant features, or by developing new features that capture important patterns in the data. Tables 2A-2H present names, weights, and relative effects ratios of top features for the four different state/outcome models developed.

TABLE 2A
Top Features for Predicting Survival
in mHSPC State (Positive Weights)

| feature_name | feature_weight | relative_effects_ratio |
|---|---|---|
| lip_Cer_Cer(d18:1/14:0)_low | 0.723 | 9.04% |
| lip_DG_DG(18:0_20:4)_low | 0.556 | 6.95% |
| lip_Cer_Cer(d18:2/22:0)_high | 0.512 | 6.41% |
| lip_Cer_Cer(d20:1/22:0)_low | 0.465 | 5.81% |
| lip_Cer_Cer(d17:1/20:0)_high | 0.434 | 5.43% |
| lip_DG_DG(18:1_18:2)_low | 0.412 | 5.16% |
| lip_Cer_Cer(d18:1/26:0)_high | 0.412 | 5.15% |
| lip_Cer_Cer(d16:1/24:1)_high | 0.407 | 5.10% |
| lip_Cer_Cer(d19:1/24:1)_high | 0.392 | 4.91% |
| lip_Cer_Cer(d18:1/22:0)_high | 0.384 | 4.81% |
| lip_Cer_Cer(m18:0/20:0)_low | 0.375 | 4.69% |
| lip_Cer_Cer(d20:1/23:0)_low | 0.366 | 4.57% |
| lip_DG_DG(18:2_18:2)_low | 0.359 | 4.49% |
| lip_DG_DG(18:1_20:3)_high | 0.357 | 4.47% |
| lip_Cer_Cer(d16:1/16:0)_low | 0.336 | 4.21% |
| lip_DG_DG(16:0_16:0)_low | 0.319 | 3.99% |
| lip_Cer_Cer(m18:1/20:0)_high | 0.31 | 3.87% |
| lip_Cer_Cer(d17:1/24:1)_high | 0.302 | 3.78% |
| gene_CHEK2_Mut | 0.29 | 3.62% |
| lip_DG_DG(14:0_18:2)_high | 0.284 | 3.55% |

TABLE 2B
Top Features for Predicting Survival
in mHSPC State (Negative Weights)

| feature_name | feature_weight | relative_effects_ratio |
|---|---|---|
| lip_Cer_Cer(d18:1/14:0)_high | −0.717 | 9.10% |
| lip_DG_DG(18:0_20:4)_high | −0.55 | 6.99% |
| lip_Cer_Cer(d18:2/22:0)_low | −0.507 | 6.43% |
| lip_Cer_Cer(d20:1/22:0)_high | −0.459 | 5.83% |
| lip_Cer_Cer(d17:1/20:0)_low | −0.428 | 5.44% |
| lip_DG_DG(18:1_18:2)_high | −0.407 | 5.16% |
| lip_Cer_Cer(d18:1/26:0)_low | −0.406 | 5.16% |
| lip_Cer_Cer(d16:1/24:1)_low | −0.402 | 5.10% |
| lip_Cer_Cer(d19:1/24:1)_low | −0.387 | 4.91% |
| lip_Cer_Cer(d18:1/22:0)_low | −0.379 | 4.81% |
| lip_Cer_Cer(m18:0/20:0)_high | −0.369 | 4.69% |
| lip_Cer_Cer(d20:1/23:0)_high | −0.36 | 4.57% |
| lip_DG_DG(18:2_18:2)_high | −0.354 | 4.49% |
| lip_DG_DG(18:1_20:3)_low | −0.352 | 4.46% |
| lip_Cer_Cer(d16:1/16:0)_high | −0.331 | 4.20% |
| lip_DG_DG(16:0_16:0)_high | −0.314 | 3.98% |
| lip_Cer_Cer(m18:1/20:0)_low | −0.304 | 3.86% |
| lip_Cer_Cer(d17:1/24:1)_low | −0.297 | 3.76% |
| lip_DG_DG(14:0_18:2)_low | −0.278 | 3.53% |
| lip_DG_DG(18:2_22:6)_high | −0.278 | 3.53% |

TABLE 2C
Top Features for Predicting Early ADT Failure
in mHSPC State (Positive Weights)

| feature_name | feature_weight | relative_effects_ratio |
|---|---|---|
| lip_DG_DG(16:0_22:6)_low | 0.880 | 11.45% |
| lip_DG_DG(18:2_20:4)_high | 0.638 | 8.31% |
| lip_DG_DG(18:1_18:2)_high | 0.632 | 8.22% |
| lip_DG_DG(16:1_18:1)_high | 0.548 | 7.14% |
| lip_DG_DG(18:0_20:4)_low | 0.428 | 5.57% |
| lip_DG_DG(18:1_20:3)_high | 0.415 | 5.40% |
| lip_DG_DG(18:1_18:1)_low | 0.396 | 5.15% |
| lip_DG_DG(16:0_18:2)_low | 0.392 | 5.10% |
| lip_DG_DG(14:0_18:2)_high | 0.390 | 5.08% |
| lip_DG_DG(18:2_22:6)_high | 0.369 | 4.80% |
| gene_BRCA2_Mut | 0.347 | 4.51% |
| lip_DG_DG(18:0_22:6)_high | 0.342 | 4.46% |
| lip_DG_DG(18:1_22:6)_high | 0.305 | 3.97% |
| lip_DG_DG(16:0_16:1)_low | 0.252 | 3.28% |
| lip_DG_DG(18:0_18:1)_low | 0.244 | 3.18% |
| lip_DG_DG(18:1_18:3)_low | 0.207 | 2.69% |
| lip_DG_DG(18:1_20:4)_low | 0.183 | 2.39% |
| lip_DG_DG(18:1_20:5)_high | 0.182 | 2.37% |
| lip_DG_DG(14:0_16:0)_high | 0.145 | 1.89% |
| lip_DG_DG(16:0_16:0)_high | 0.139 | 1.81% |

TABLE 2D
Top Features for Predicting Early ADT Failure
in mHSPC State (Negative Weights)

| feature_name | feature_weight | relative_effects_ratio |
|---|---|---|
| gene_CHEK2_SNP | −1.127 | 13.19% |
| lip_DG_DG(16:0_22:6)_high | −0.852 | 9.97% |
| gene_ATM_Mut | −0.727 | 8.50% |
| lip_DG_DG(18:2_20:4)_low | −0.610 | 7.14% |
| lip_DG_DG(18:1_18:2)_low | −0.604 | 7.06% |
| lip_DG_DG(16:1_18:1)_low | −0.520 | 6.09% |
| lip_DG_DG(18:0_20:4)_high | −0.400 | 4.68% |
| lip_DG_DG(18:1_20:3)_low | −0.387 | 4.53% |
| lip_DG_DG(18:1_18:1)_high | −0.367 | 4.30% |
| lip_DG_DG(16:0_18:2)_high | −0.363 | 4.25% |
| lip_DG_DG(14:0_18:2)_low | −0.362 | 4.24% |
| lip_DG_DG(18:2_22:6)_low | −0.341 | 3.99% |
| lip_DG_DG(18:0_22:6)_low | −0.314 | 3.68% |
| lip_DG_DG(18:1_22:6)_low | −0.277 | 3.24% |
| lip_DG_DG(16:0_16:1)_high | −0.224 | 2.62% |
| lip_DG_DG(18:0_18:1)_high | −0.216 | 2.53% |
| lip_DG_DG(18:1_18:3)_high | −0.179 | 2.09% |
| lip_DG_DG(18:1_20:4)_high | −0.155 | 1.82% |
| lip_DG_DG(18:1_20:5)_low | −0.154 | 1.81% |

TABLE 2E
Top Features for Early Death Prediction
in mCRPC State (Positive Weights)

| feature_name | feature_weight | relative_effects_ratio |
|---|---|---|
| gene_RB1_Del | 2.208 | 15.37% |
| lip_Cer_Cer(d18:1/17:0)_low | 0.699 | 4.87% |
| lip_Cer_Cer(d20:1/23:0)_high | 0.681 | 4.74% |
| lip_Sph_Sph(d16:1)_low | 0.592 | 4.12% |
| lip_Cer_Cer(d18:1/24:1)_high | 0.537 | 3.74% |
| lip_Cer_Cer(d17:1/24:1)_high | 0.53 | 3.69% |
| lip_Cer_Cer(d19:1/24:0)_low | 0.527 | 3.67% |
| lip_Cer_Cer(d17:1/18:0)_low | 0.511 | 3.56% |
| lip_Cer_Cer(d20:1/22:0)_high | 0.504 | 3.51% |
| lip_Cer_Cer(m18:1/18:0)_high | 0.5 | 3.48% |
| lip_Cer_Cer(d16:1/20:0)_low | 0.481 | 3.35% |
| lip_Cer_Cer(d18:1/26:0)_low | 0.461 | 3.21% |
| lip_Sph_Sph(d18:1)_high | 0.458 | 3.19% |
| gene_AR_Amp | 0.442 | 3.08% |
| lip_Cer_Cer(m18:1/22:0)_high | 0.436 | 3.04% |
| lip_Cer_Cer(d18:1/20:0)_high | 0.432 | 3.01% |
| lip_Cer_Cer(d18:1/21:0)_low | 0.401 | 2.79% |
| lip_Cer_Cer(d19:1/26:0)_high | 0.394 | 2.74% |
| lip_Cer_Cer(d18:1/24:0)_low | 0.393 | 2.74% |
| lip_Cer_Cer(m18:0/20:0)_low | 0.376 | 2.62% |

TABLE 2F
Top Features for Early Death Prediction
in mCRPC State (Negative Weights)

| feature_name | feature_weight | relative_effects_ratio |
|---|---|---|
| lip_Cer_Cer(d18:1/17:0)_high | −0.741 | 5.45% |
| lip_Cer_Cer(d20:1/23:0)_low | −0.723 | 5.31% |
| lip_Sph_Sph(d16:1)_high | −0.634 | 4.66% |
| lip_Cer_Cer(d18:1/24:1)_low | −0.579 | 4.26% |
| lip_Cer_Cer(d17:1/24:1)_low | −0.572 | 4.20% |
| lip_Cer_Cer(d19:1/24:0)_high | −0.569 | 4.18% |
| lip_Cer_Cer(d17:1/18:0)_high | −0.553 | 4.07% |
| lip_Cer_Cer(d20:1/22:0)_low | −0.546 | 4.01% |
| lip_Cer_Cer(m18:1/18:0)_low | −0.541 | 3.98% |
| lip_Cer_Cer(d16:1/20:0)_high | −0.523 | 3.84% |
| lip_Cer_Cer(d18:1/26:0)_high | −0.503 | 3.70% |
| lip_Sph_Sph(d18:1)_low | −0.500 | 3.68% |
| gene_TP53_Mut | −0.479 | 3.52% |
| lip_Cer_Cer(m18:1/22:0)_low | −0.478 | 3.52% |
| lip_Cer_Cer(d18:1/20:0)_low | −0.474 | 3.48% |
| lip_Cer_Cer(d18:1/21:0)_high | −0.443 | 3.25% |
| lip_Cer_Cer(d19:1/26:0)_low | −0.436 | 3.21% |
| lip_Cer_Cer(d18:1/24:0)_high | −0.435 | 3.20% |
| lip_Cer_Cer(m18:0/20:0)_high | −0.417 | 3.07% |
| lip_Cer_Cer(d16:1/22:0)_high | −0.414 | 3.04% |

TABLE 2G
Top Features for Late Death Prediction
in mCRPC State (Positive Weights)

| feature_name | feature_weight | relative_effects_ratio |
|---|---|---|
| lip_Cer_Cer(d19:1/26:0)_low | 0.686 | 6.54% |
| lip_Cer_Cer(d19:1/24:0)_high | 0.543 | 5.18% |
| lip_Cer_Cer(d18:1/24:0)_high | 0.523 | 4.99% |
| lip_Cer_Cer(d18:2/24:0)_low | 0.521 | 4.97% |
| lip_Cer_Cer(d18:1/17:0)_high | 0.492 | 4.70% |
| lip_Cer_Cer(d20:1/23:0)_low | 0.488 | 4.65% |
| lip_Cer_Cer(d18:1/22:0)_high | 0.453 | 4.32% |
| lip_Cer_Cer(d18:1/24:1)_low | 0.440 | 4.19% |
| lip_Cer_Cer(d19:1/18:0)_high | 0.420 | 4.01% |
| lip_Cer_Cer(d19:1/20:0)_low | 0.404 | 3.86% |
| lip_Cer_Cer(d18:1/14:0)_low | 0.398 | 3.79% |
| lip_Cer_Cer(d18:2/24:1)_high | 0.393 | 3.74% |
| lip_Cer_Cer(d16:1/24:0)_high | 0.355 | 3.39% |
| lip_Cer_Cer(d16:1/23:0)_high | 0.347 | 3.31% |
| lip_Cer_Cer(m18:0/22:0)_high | 0.331 | 3.16% |
| lip_Cer_Cer(d17:1/23:0)_low | 0.329 | 3.13% |
| lip_Cer_Cer(d18:1/26:0)_high | 0.327 | 3.12% |
| lip_Cer_Cer(m18:1/24:1)_high | 0.301 | 2.87% |
| lip_Cer_Cer(d18:1/19:0)_low | 0.299 | 2.86% |
| lip_Cer_Cer(d16:1/18:0)_high | 0.281 | 2.68% |

TABLE 2H
Top Features for Late Death Prediction
in mCRPC State (Negative Weights)

| feature_name | feature_weight | relative_effects_ratio |
|---|---|---|
| gene_AR_Amp | −0.940 | 7.49% |
| gene_RB1_Del | −0.850 | 6.78% |
| lip_Cer_Cer(d19:1/26:0)_high | −0.698 | 5.56% |
| lip_Cer_Cer(d19:1/24:0)_low | −0.555 | 4.42% |
| lip_Cer_Cer(d18:1/24:0)_low | −0.535 | 4.26% |
| lip_Cer_Cer(d18:2/24:0)_high | −0.533 | 4.25% |
| lip_Cer_Cer(d18:1/17:0)_low | −0.505 | 4.02% |
| lip_Cer_Cer(d20:1/23:0)_high | −0.500 | 3.98% |
| gene_TP53_Mut | −0.485 | 3.86% |
| lip_Cer_Cer(d18:1/22:0)_low | −0.465 | 3.71% |
| lip_Cer_Cer(d18:1/24:1)_high | −0.452 | 3.60% |
| lip_Cer_Cer(d19:1/18:0)_low | −0.432 | 3.44% |
| lip_Cer_Cer(d19:1/20:0)_high | −0.417 | 3.32% |
| lip_Cer_Cer(d18:1/14:0)_high | −0.410 | 3.27% |
| lip_Cer_Cer(d18:2/24:1)_low | −0.405 | 3.23% |
| lip_Cer_Cer(d16:1/24:0)_low | −0.368 | 2.93% |
| lip_Cer_Cer(d16:1/23:0)_low | −0.359 | 2.86% |
| lip_Cer_Cer(m18:0/22:0)_low | −0.344 | 2.74% |
| lip_Cer_Cer(d17:1/23:0)_high | −0.341 | 2.72% |
| lip_Cer_Cer(d18:1/26:0)_low | −0.339 | 2.70% |

Next, we adopted the survival prediction task for mHSPC and mCRPC patients to compute the test accuracy of the multi-omic model that included both genomic and lipidomic features, and then compared our method with a previously published candidate-based, three-lipid-signature approach (See Lin, H. M., et al., Aberrations in circulating ceramide levels are associated with poor clinical outcomes across localised and metastatic prostate cancer. Prostate Cancer Prostatic Dis, 2021) which was used for predicting clinical outcomes.

The three-lipid-signature only uses three lipid species (ceramide (d18:1/24:1), sphingosine (d18:2/16:0), and phosphatidylcholine (16:0/16:0)) which limits the model's capacity relative to the disclosed method. A comparation between the disclosed method and three-signature method is made. We adopt the survival prediction task over mHSPC patients as the target. To make a fair comparison, we compared the proposed machine learning method with the standard three-lipids-model, a three-lipids-model with gene features in mHSPC (mutations in ATM, BRCA1, BRCA2, CHEK2) and a three-lipids-model with bin-split binarized lipid marker features. We randomly select 60% of patients' data for model training and evaluate with the remaining 40%. For each approach, we compute the true-positive-rate (TPR) and false-positive-rate (FPR) and then draw the ROC curve. We compute the area under the ROC curve (i.e., C-index), as the final evaluation metric. Results are shown in Table 3.

TABLE 3
AUC (C-index) Values for Disclosed Method
vs. Conventional Three Lipid-Based Methods

| mHSPC-Survival | AUC (C-index) |
|---|---|
| Three Lipid Signatures | 0.56 |
| Three Lipid Signatures with Gene features (mutations | 0.58 |
| in ATM, BRCA1, BRCA2, CHEK2) | |
| Three Lipid Signatures with Bin-split binarization | 0.52 |
| Multi-omics features with Bin-split binarization (presently | 0.71 |
| disclosed method) | |

### Outcome Predictions Using the Predictive Model

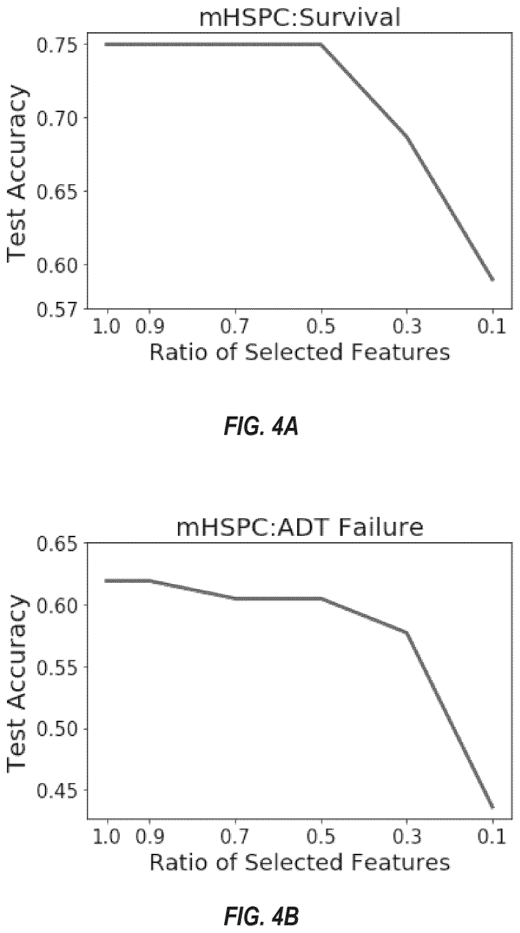

FIG. 3 illustrates an example approach for calculating a probability score using a limited number of features (i.e., a limited number of feature weights generated by the machine learning process and a limited number of corresponding patient features).

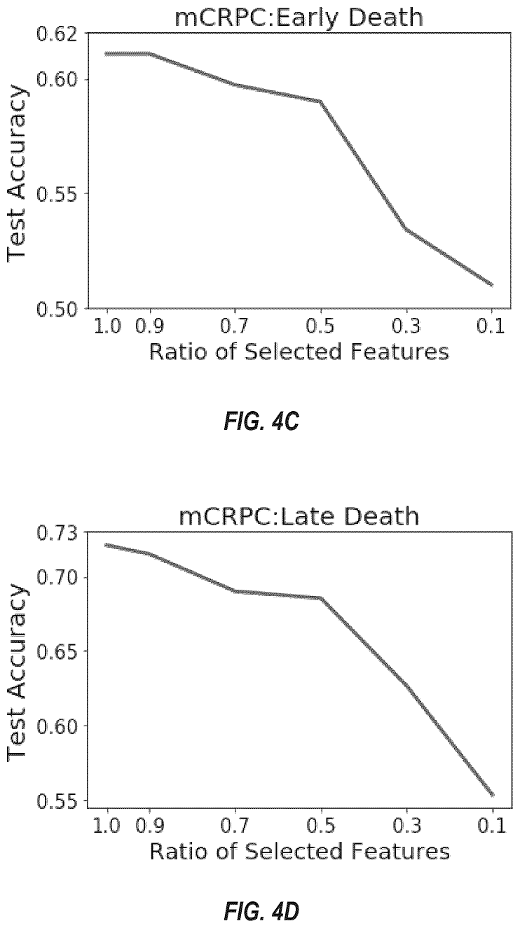

We also investigated the trade-off effects between the number of features used and the sacrifice of the prediction performance by only picking up the top N features with largest effects. Results are shown in FIGS. 4A-4D, which illustrate test accuracy as a function of the ratio of selected features (N) to total candidate features of the training dataset. The test accuracy scores were calculated as the mean of 5-folds cross validation. As shown, high accuracy is maintained even when we consider only 50% of all features of the original training dataset. This sparsity effect allows investigation/measurement of fewer patient features, decreases costs, and maintains accuracy of prediction probabilities for potential clinical applications.

For the mHSPC survival model, the number of features in the training dataset was 176, including genetic markers in ATM, BRCA1, BRCA2, and CHECK2, and lipid markers in ceramide lipid species and diacylglycerol lipid species. As shown in FIG. 4A, a test accuracy of 0.75 was maintained even when we used a 0.5 ratio (i.e., when half of the 176 features were used in the predictive model).

For the mHSPC ADT failure model, the number of features in the training dataset was 54, including genetic markers in ATM, BRCA1, BRCA2, and CHECK2, and lipid markers in diacylglycerol lipid species. As shown in FIG. 4B, a test accuracy of 0.6 was maintained even when we used a 0.5 ratio (i.e., when half of the 54 features were used in the predictive model).

For the mCRPC early death model, the number of features in the training dataset was 131, including genetic markers related to TP53 mutation, RB1 loss, and AR alterations, and lipid markers in ceramide lipid species and sphingoid lipid species. As shown in FIG. 4C, test accuracy of 0.6 was substantially maintained even when we used a 0.5 ratio (i.e., when half of the 131 features were used in the predictive model).

For the mCRPC late death model, the number of features in the training dataset was 125, including genetic markers related to TP53 mutation, RB1 loss, and AR alterations, and lipid markers in ceramide, sphingoid, and acylcarnitine lipid species. As shown in FIG. 4D, test accuracy of 0.7 was substantially maintained even when we used a 0.5 ratio (i.e., when half of the 125 features were used in the predictive model).

Based on the success of the reduced feature ratios, the predictive model was implemented to calculate the PS of the four clinical outcomes, for each relevant patient in the cohort, using a limited number of features. Four examples are illustrated below, one for each of the clinical outcome models:

Subject ID: 1028, Probability Score Calculation: 1/(1+exp (−(0.884*1+−0.874*0+0.624*1+−0.613*0+0.564*1+−0.554*0+0.523*0+−0.513*1+0.498*0+0.495*1+0.489*1+−0.488*1+−0.485*0+−0.478*0+0.474*0+−0.464*1+0.459*0+−0.449*1+0.441*0+−0.431*1+0.417*0+−0.407*1+0.378*0+−0.368*1+0.355*1+0.351*0+−0.345*0+0.341*1+−0.341*1+0.340*1+0.340*1+−0.331*0+−0.329*0+−0.329*0+0.319*1+−0.309*0+0.295*0+0.293*0+−0.285*1+−0.283*1+0.280*1+0.276*0+0.274*1+−0.270*0+−0.266*1+−0.264*0+0.260*0+0.257*0+0.253*0+0.250*0+−0.250*1+−0.247*1+0.247*1+0.243*0+−0.242*1+0.242*1+0.241*0+−0.240*1+−0.237*0+0.234*0+−0.233*1+−0.232*0+0.231*0+−0.230*1+0.225*0+−0.221*1+−0.215*1+0.211*0+0.203*1+0.202*1+−0.201*1+−0.193*0+0.193*0+0.192*1+−0.192*0+0.189*1+0.186*1+−0.182*1+−0.182*0+0.180*1+−0.179*0+−0.176*0+0.174*1+−0.170*0+0.166*1+0.166*1+−0.163*0+0.162*0)))=0.710 (71.0%) that subject with mHSPC will survive.

Subject ID: 1028, Probability Score: 1/(1+exp (−(−1.106*0+−1.023*0+0.739*0+0.736*1+−0.711*1+−0.708*0+0.587*0+−0.559*1+0.554*0+−0.527*1+0.477*0+0.454*0+−0.450*1+−0.426*1+0.413*1+0.392*0)))=0.179 (17.9%) for predicting subject will have early (within 6 months) ADT failure.

Subject ID: 1001, Probability Score calculation: 1/(1+exp (−(2.460*0+−0.790*1+−0.742*0+0.737*0+−0.719*0+0.689*1+−0.685*1+0.666*1+−0.659*0+−0.640*1+0.632*0+0.606*1+−0.597*1+0.587*0+−0.569*0+0.544*0+−0.540*1+−0.536*0+−0.522*0+0.516*1+−0.489*0+0.487*0+0.483*1+−0.478*0+0.469*1+−0.454*1+−0.448*1+−0.446*0+0.436*1+−0.429*0+0.425*1+0.424*0+0.401*0+0.395*0+0.393*1+−0.390*1+0.376*1+−0.371*1+−0.360*1+0.336*0+−0.331*0+−0.328*1+−0.323*0+0.318*0+−0.317*1+−0.307*1+0.307*0+−0.306*1+−0.291*1+0.278*1+0.275*0+−0.275*1+0.270*1+0.264*0+−0.263*1+−0.259*0+0.255*0+0.253*0+−0.251*0+0.239*0+−0.235*0+−0.227*1+−0.226*1+−0.224*0+0.222*0)))=0.099 (9.9%) for predicting subject will have poor prognosis in mCRPC state.

Subject ID: 1001, Probability Score calculation: 1/(1+exp (−(−1.075*0+−0.830*0+−0.807*0+0.794*1+−0.710*1+0.696*0+−0.669*0+−0.629*0+0.616*1+−0.601*1+0.588*0+−0.580*1+0.567*0+−0.529*0+0.515*1+−0.510*1+0.496*0+−0.481*0+−0.476*0+−0.471*1+0.468*1+0.463*1+0.458*0+−0.457*0+−0.451*1+0.444*1+0.439*0+−0.435*0+0.422*1+−0.406*1+0.392*0+−0.380*0+−0.371*1+−0.371*0+−0.371*0+0.367*1+0.358*0+0.358*1+0.358*1+−0.351*0+−0.347*1+−0.338*1+0.338*1+−0.335*0+0.333*0+0.325*0+0.322*1+−0.303*1+0.290*0+−0.290*1+−0.285*0+0.277*0+0.272*1+−0.269*0+−0.260*0+−0.260*1+0.256*1+0.247*1+0.247*0+−0.232*0+−0.230*0+−0.224*0+−0.221*0+0.219*1+0.216*1+0.211*1+0.208*1+−0.182*1+−0.174*0+−0.172*1+0.169*0+−0.167*1+0.161*1+−0.160*0+0.159*0+−0.157*0+−0.153*1+0.153*0+−0.153*1)))=0.688 (68.8%) that mCRPC subject will have exceptional survival.

### Computer System Details

The methods disclosed herein can be carried out at least in part using one or more computer systems. Such computer systems can include one or more processors and computer-readable media such as computer memory stored on one or more hardware storage devices. The computer memory may store computer-executable instructions that when executed by one or more processors cause various functions to be performed, such as the acts recited herein.

Computer-readable media can include any media that can be accessed by the controller. Physical computer-readable storage media includes RAM, ROM, EEPROM, optical or magnetic storage devices, or any other medium which can be used to store desired program code means in the form of computer-executable instructions or data structures and which can be accessed by a computer.

Computer functionality can additionally or alternatively be carried out by one or more hardware logic components. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), and the like.

The computer system may be interconnected to one or more other computer systems via one or more network connections. Network connections may include, but are not limited to, connections via wired or wireless Ethernet, cellular connections, or even computer to computer connections through serial, parallel, USB, or other connections. The computer system may be included in a distributed system environment in which local and remote computer systems, which are linked (either by hardwired data links, wireless data links, or by a combination of hardwired and wireless data links) through a network, both perform tasks. In a distributed system environment, program modules may be located in both local and remote memory storage devices.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

The various features of a given embodiment can be combined with and/or incorporated into other embodiments disclosed herein. Thus, disclosure of certain features relative to a specific embodiment of the present disclosure should not be construed as limiting application or inclusion of said features to the specific embodiment. Rather, it will be appreciated that other embodiments can also include such features.

In addition, unless otherwise indicated, numbers expressing quantities, constituents, distances, or other measurements used in the specification and claims are to be understood as optionally being modified by the term “about.” When the terms “about,” “approximately,” “substantially,” or the like are used in conjunction with a stated amount, value, or condition, it may be taken to mean an amount, value or condition that deviates by less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

The embodiments disclosed herein should be understood as comprising/including disclosed components, and may therefore include additional components not specifically described. Optionally, the embodiments disclosed herein are essentially free or completely free of components that are not specifically described. That is, non-disclosed components may optionally be completely omitted or essentially omitted from the disclosed embodiments. For example, genetic or lipid markers that are not specifically disclosed herein may optionally be expressly omitted.

1. A method for predicting a clinical outcome in a metastatic prostate cancer patient based on measurements of multi-omic features of the patient, the method comprising:
selecting a weighting model based on (i) a metastatic prostate cancer state and (ii) a clinical outcome of interest, the weighting model mapping weights to a series of features with respect to the clinical outcome of interest, wherein the features comprise lipid markers and genetic markers, and wherein the weights are generated using a machine learning process that utilizes a training dataset that associates the features of multiple historic metastatic prostate cancer patients with recorded clinical outcomes of those patients; and
determining a probability score (PS) for the patient according to a predictive model defined by:
$$PS = \frac{1}{1 + exp ( - {\sum }_{n = 1}^{N} {w}_{n} {x}_{n} )}$$
wherein PS represents the likelihood of occurrence of the clinical outcome of interest, N is the number of different features used in the determination of PS, wₙ are weights for each nth feature as provided by the selected weighting model, and x, are the measured values of the patient for each nth feature.

2. The method of claim 1, wherein:
the state of the patient is a metastatic hormone sensitive prostate cancer (mHSPC) state, and the clinical outcome of interest is: (i) survival in the mHSPC state or (ii) androgen deprivation therapy (ADT) failure in the mHSPC state, defined as failure within a predetermined time period following initiation of ADT, or
the state of the patient is a metastatic castrate resistant prostate cancer (mCRPC) state, and the clinical outcome of interest is (i) early death in the mCRPC state, or (ii) long-term survival in the mCRPC state.

3. The method of claim 2, wherein early death in the mCRPC state is defined as being within a predetermined bottom quantile or set of quantiles for survival time following progression to the mCRPC state, and wherein long-term survival in the mCRPC state is defined as being within a predetermined top quantile or set of quantiles for survival time following progression to the mCRPC state.

4. The method of claim 2, wherein:
the patient is an mHSPC patient;
the predictive model is configured for predicting ADT failure;
the PS for the patient is above a predetermined threshold value, thereby indicating likelihood that ADT failure will occur early; and
the patient treatment plan is adapted to account for the PS by incorporating additional or alternative treatments.

5. The method of claim 2, wherein:
the patient is an mCRPC patient;
the predictive model is configured for predicting early death in the mCRPC state or is configured for predicting late death in the mCRPC state; and
the patient treatment plan is adapted to account for the PS by:
promoting more aggressive treatments when the PS indicates high probability of early death and/or low probability of long-term survival, or
minimizing more aggressive treatments to thereby minimize side effects when the PS indicates low probability of early death and/or high probability of long-term survival.

6. The method of claim 1, wherein a patient treatment plan is developed or modified based on the determined probability score.

7. The method of claim 1, wherein the lipid markers comprise plasma lipid markers.

8. The method of claim 1, wherein the genetic markers comprise circulating tumor DNA (ctDNA) markers.

9. The method of claim 1, wherein the genetic markers comprise one or more of ATM, BRCA1, BRCA2, or CHEK2 when the patient is in the mHSPC state.

10. The method of claim 1, wherein the genetic markers comprise one or more of TP53, RB1 loss, or AR amplification when the patient is in the mCRPC state.

11. The method of claim 1, wherein the lipid markers comprise one or more of ceramide (Cer) species, diacylglycerol (DG) species, and triacylglycerol (TG) species when the patient is in the mHSPC state.

12. The method of claim 1, wherein the lipid markers comprise one or more of Cer species, sphingosine (Sph) species, and acylcarnitine (Acy) species when the patient is in the mCRPC state.

13. The method of claim 1, wherein the genetic markers are binarized as present or absent.

14. The method of claim 1, wherein the lipid markers are binarized using a bin-split binarization process in which each lipid marker measurement is mapped to a high-level binary feature and a low-level binary feature, wherein for each lipid marker a predetermined threshold level is used as a cutoff between the high-level binary feature and the low-level binary feature for that lipid marker.

15. The method of claim 14, wherein the predetermined threshold level for each lipid marker is the median or mean level of that lipid marker in the training dataset.

16. The method of claim 1, wherein the machine learning process comprises one or more of: logistic regression, optionally with elastic-net regularization; kernel support vector machines (kernel-SVM); or Gaussian process regression (GPR).

17. The method of claim 1, wherein given a training dataset with at least about 50 features, N is lowerable to as low as 50% of the total number of features in the training dataset without substantial degradation in accuracy of the resulting PS.

18. The method of claim 17, wherein the N number of features used in the determination of PS are the N number of features with the highest effect sizes as determined by the machine learning process.

19. A method for predicting a clinical outcome in a metastatic prostate cancer patient based on measurements of multi-omic features of the patient, the method comprising:
selecting a weighting model based on a metastatic prostate cancer state and a clinical outcome of interest, the weighting model comprising
(i) a model for predicting survival in a metastatic hormone sensitive prostate cancer (mHSPC) state,
(ii) a model for predicting androgen deprivation therapy (ADT) failure in the mHSPC state, defined as failure within a predetermined time period following initiation of ADT,
(iii) a model for predicting early death in a metastatic castrate resistant prostate cancer (mCRPC) state, or
(iv) a model for predicting long-term survival in the mCRPC state,
wherein the weighting model maps weights to a series of features with respect to the corresponding clinical outcome, wherein the features comprise plasma lipid markers and genetic markers, the genetic markers comprising circulating tumor DNA (ctDNA) markers, and
wherein the weights are generated using a machine learning process that utilizes a training dataset that associates the features of multiple historic metastatic prostate cancer patients with recorded clinical outcomes of those patients; and
determining a probability score (PS) for the patient according to a predictive model defined by:
$$PS = \frac{1}{1 + exp ( - {\sum }_{n = 1}^{N} {w}_{n} {x}_{n} )}$$
wherein PS represents the likelihood of occurrence of the clinical outcome, N is the number of different features used in the determination of PS, wₙ are weights for each nth feature as provided by the selected weighting model, and x, are the measured values of the patient for each nth feature,
wherein the genetic markers are binarized as present or absent, and wherein the lipid markers are binarized using a bin-split binarization process in which each lipid marker measurement is mapped to a high-level binary feature and a low-level binary feature, wherein for each lipid marker a predetermined threshold level is used as a cutoff between the high-level binary feature and the low-level binary feature for that lipid marker.

20. A computer system configured to generate a probability score for a clinical outcome in a metastatic prostate cancer patient based on measurements of multi-omic features of the patient, the computer system comprising:
one or more processors; and
one or more hardware storage devices comprising computer-executable instructions stored thereon that are executable by the one or more processors to cause the computer system to at least:
receive a first input selecting a metastatic prostate cancer state and to a clinical outcome of interest;
incorporating the first input into a weighting model, the weighting model mapping weights to a series of features with respect to the clinical outcome of interest, wherein the features comprise lipid markers and genetic markers, and wherein the weights are generated using a machine learning process that utilizes a training dataset that associates the features of multiple historic metastatic prostate cancer patients with recorded clinical outcomes of those patients;
receive a second input comprising measured values of a patient for the features, including measured values of the patient for the lipid markers and genetic markers;
determine a probability score (PS) for the patient according to a predictive model defined by:
$$PS = \frac{1}{1 + exp ( - {\sum }_{n = 1}^{N} {w}_{n} {x}_{n} )}$$
wherein PS represents the likelihood of occurrence of the clinical outcome of interest, N is the number of different features used in the determination of PS, wₙ are weights for each nth feature as provided by the weighting model, and xₙ are the measured values of the patient for each nth feature; and
output the PS to enable development or modification of a treatment plan for the patient.
